(19) United States
(12) Patent Application Publication
(10) Pub. No.: US 2002/0198544 A1
(43) Pub. Date: Dec. 26, 2002

(54) Percutaneous facelift device

(76) Inventor: Renan Uflacker, Mt. Pleasant, SC (US)

Correspondence Address: David L. Principe, Hodgson Russ LLP, Suite 2000, One M&T Plaza, Buffalo, NY, 14203-2391, US

(21) Appl. No.: 10/178,435

(22) Filed: Jun. 24, 2002

Related U.S. Application Data
(60) Provisional application No. 60/300,146, filed on Jun. 22, 2001.

(51) Int. Cl. A61B 17/04

(52) U.S. Cl. 606/144

(57) ABSTRACT

A simplified percutaneous technique for the elevation and traction of facial, head and neck soft tissues, including the midface, malar tissues, eyebrows, nasolabial folds, labiomental folds, infraorbital hollows, neck soft tissues, and jowls.

### CROSS-REFERENCE TO RELATED APPLICATION

Applicant hereby claims priority based on U.S. Provisional Application No. 60/300,146 filed Jun. 22, 2001, entitled “Percutaneous Facelift Device” which is incorporated herein by reference.

### FIELD OF THE INVENTION

The present invention pertains to a device and method for stretching the skin and reducing the wrinkles and sagging areas of the face, head, and neck.

### BACKGROUND OF THE INVENTION

Facelift surgery is currently done with large surgical incisions and detachment of the face and forehead skin from the deeper tissues for stretching the skin and reducing the wrinkles and sagging areas of the face.

What is needed is a simplified percutaneous technique for the elevation and traction of facial, head and neck soft tissues, including the midface, malar tissues, eyebrows, nasolabial folds, labiomental folds, infraorbital hollows, neck soft tissues, and jowls.

### BRIEF DESCRIPTION OF THE DRAWINGS

The invention is illustrated in the drawings in which like reference characters designate the same or similar parts throughout the figures of which:

FIG. 1 is a perspective view showing the positioning of the device of the present invention on the head;

FIG. 2 is a perspective view of a curved needle for deployment of the device of the present invention;

FIG. 3 is a perspective view of a straight needle;

FIG. 4 is a detailed side elevational view of a needle pre-loaded with the anchoring devices of the present invention;

FIG. 5 is a schematic drawing of the anchoring device, suture, and tunneling device, of the present invention;

FIG. 6 is a detailed perspective view of the anchoring device of the present invention;

FIG. 7 is a side elevational view showing the deployment of the device in progress;

FIG. 8 is a schematic drawing of an alternate embodiment of the anchoring device, suture and tunneling device;

FIG. 9 is an enlarged partial view of a portion of the anchoring device; and,

FIG. 10 is a side elevational view showing an alternate deployment of the device.

### DETAILED DESCRIPTION

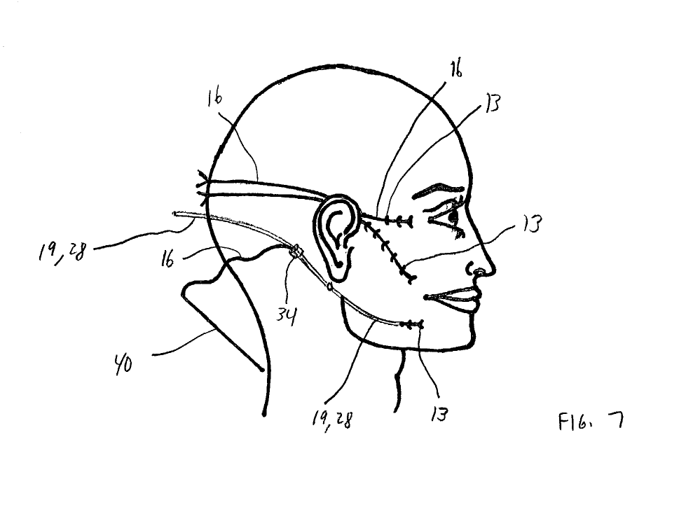

Referring generally to FIGS. 1-10, and initially to FIG. 1, a percutaneous facelift device 10 includes a plurality of anchoring devices 13 that are deployed in the subcutaneous tissues of the face. The anchoring devices 13 are flexible so that they can be initially deployed through a needle or catheter at a lower profile. Once they are deployed, the anchoring devices uncoil into their extended position (best shown in FIGS. 5-6). The anchoring devices 13 may be constructed of metal, plastic, or a bioabsorbable polymer, polymer blend, composite or the like. A reabsorbable polymer may be used to form the implantable device so that it maintains a specific proportion of the strength within the soft tissues for a period of time at least equal to the healing period, and will eventually be reabsorbed avoiding the permanent presence of implants or sutures under the skin.

The anchoring devices 13 are applied to both sides of the face and the sutures 16 holding the anchoring devices 13 are joined at the back of the head and neck where both sides are tied together pulling symmetrically on the face. The anchoring devices 13 may be deployed through a small incision in the skin behind the mandible angle of the face or other entry points as shown in FIG. 1.

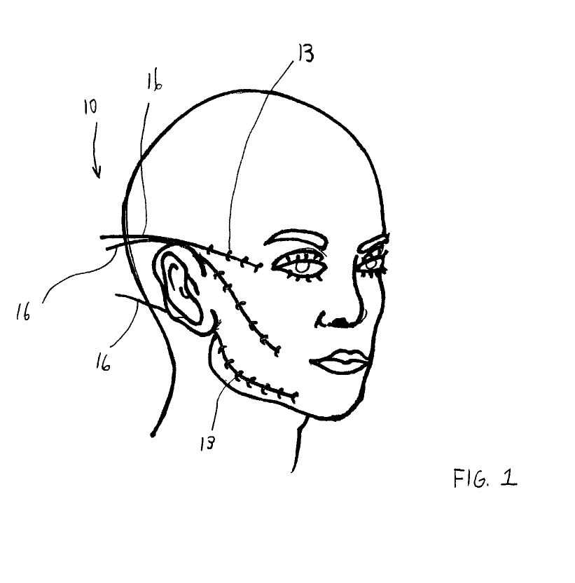

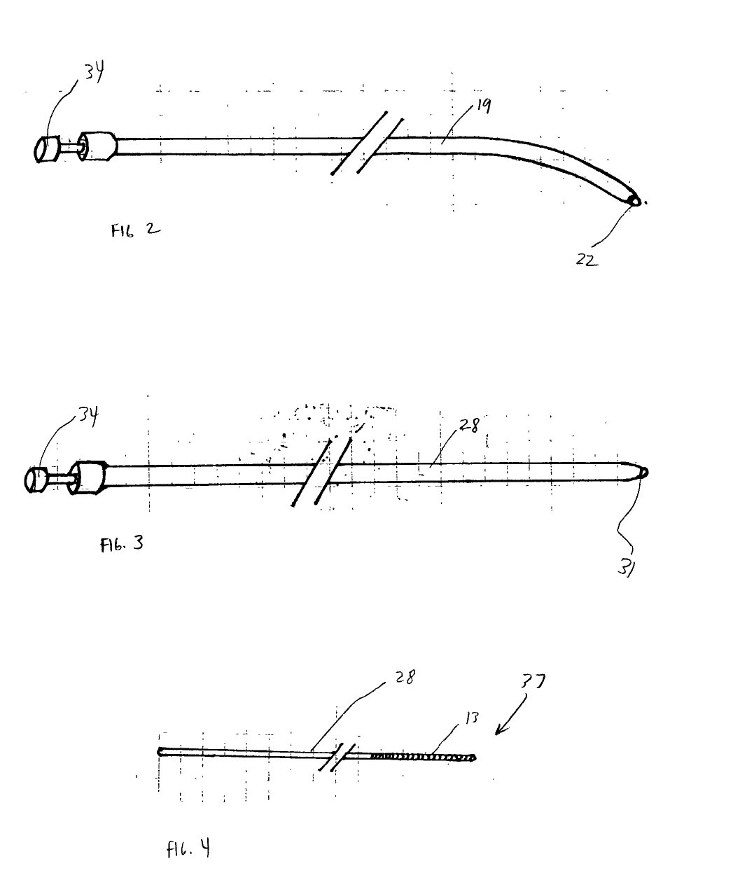

Turning to FIGS. 2 and 3, the anchoring devices 13 may be deployed through a curved needle 19 (FIG. 2) having a blunt tip end 22 or through a straight needle 28 having a blunt tip end 31. The needles 19 and 28 include an axially disposed plunger 34 that is used to deploy the anchoring devices 13. Once the needle 19, 28 has been inserted into the skin through the entry incision and has been advanced into the subcutaneous tissue where it is to be deployed, the plunger 34 is held in position while the needle 19, 28 is removed from the anchoring devices 13 by retracting the needles 19, 28. Accordingly, the anchoring devices 13 are thereby released from the needle 19, 28 into the subcutaneous tissue.

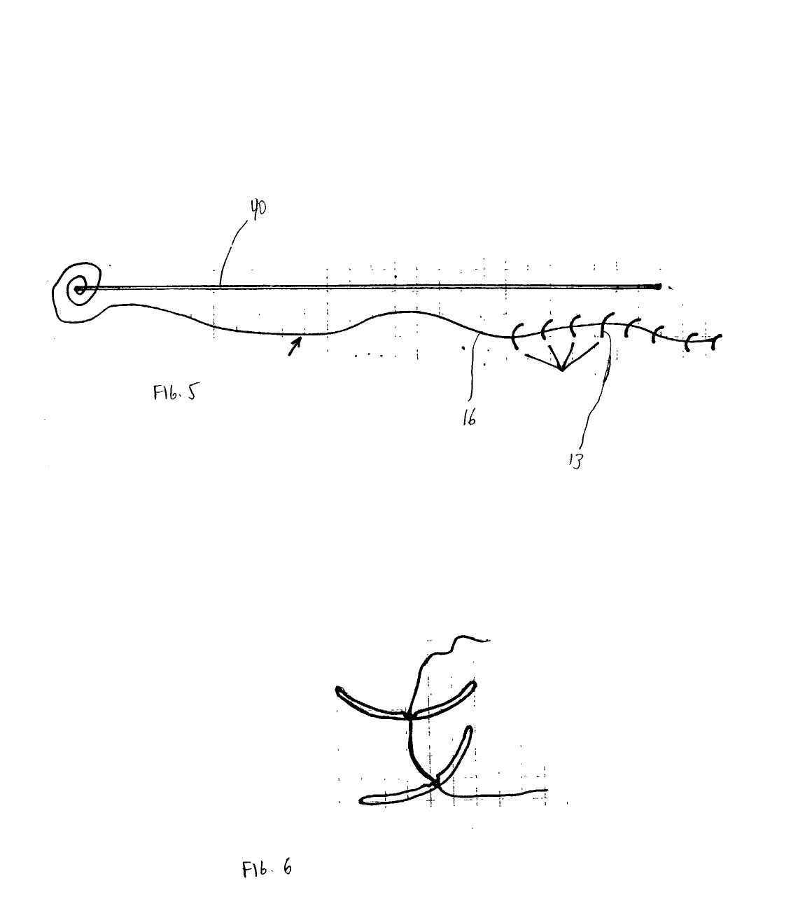

In FIG. 4, a needle 28 is shown with a plurality of anchoring devices 13 preloaded into the distal end 37 of the needle 28. The anchoring devices 13 may be preloaded into the needle 28 or via a cartridge (not shown) introduced through the needle.

Turning to FIGS. 5 and 6, the anchoring devices 13 are attached in a line by the suture 16 that is disposed along a midpoint of each anchoring device 13. The arrangement shown is one example. Other configurations of the anchoring devices 13 and sutures 16 may also be suitable. Traction of the sutures 16 on both sides of the face produces a symmetrical pull of the facial tissues.

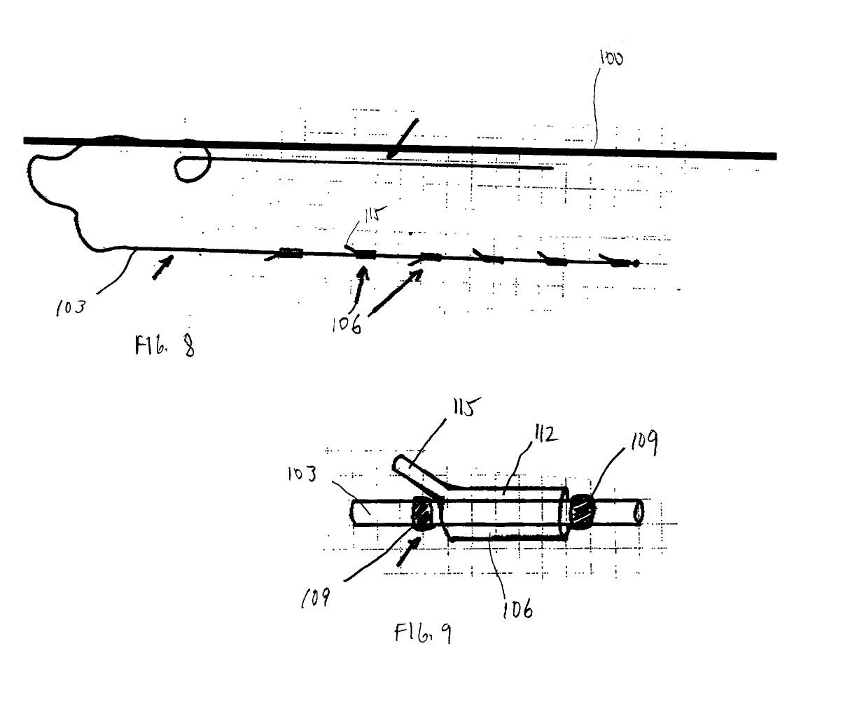

As shown in FIG. 7, The suture 16 is operatively associated with a tunneling device 40. The tunneling device 40 carries the suture to the back of the head and neck underneath the skin and is then removed leaving only a suture 16 from each side to be tied in the back, as best shown in FIG. 7. The sutures 16 holding the anchoring devices 13 are tunneled under the skin and the scalp all the way back into the head/neck. The sutures 16 from each side of the face are tunneled in this manner through the soft tissues of the base of the head and the sutures 16 from each side of the face are tied up in the back underneath the skin with proper tension for the desired facelift. The sutures 16 disposed on one side of the face are contralateral to the sutures 16 disposed on the opposite side of the face. Accordingly, contralateral suture is defined herein as the sutures disposed on the opposite side of the face. Attaching a suture to the contralateral suture results in a symmetrical pull to both sides of the face.

The device can also be applied to the temples and the forehead for lifting of the wrinkles and flabby areas.

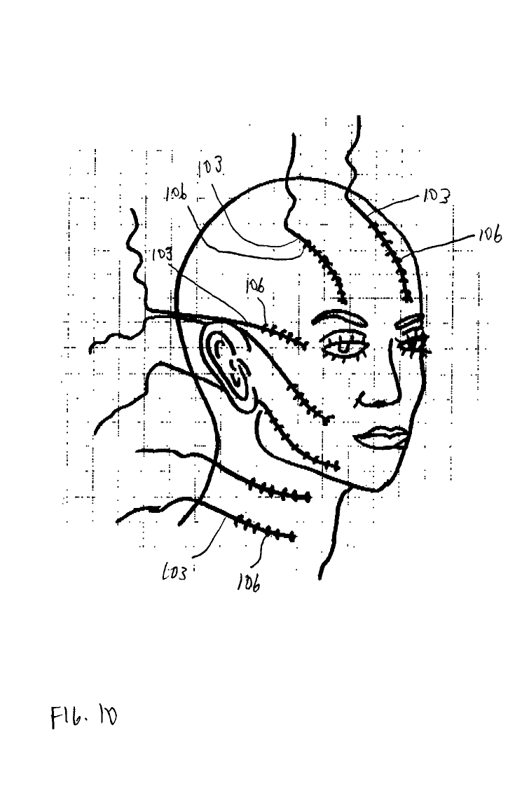

In FIGS. 8-9, an alternate needle 100 is shown. A suture 103 having an array of anchors 106 is shown. The anchors 106 are in variable number according to the area to be treated. The anchors 106 can be made of metal, plastic or polymer and may or may not be absorbable. The needle 100 is deployed to the target region subcutaneously and then the anchors 106 are deployed by retracting the needle 100 from the suture 103. As known to those of ordinary skill in the art, the needle 100 positively engages with the anchors 106 by means of a plunger or other mechanical device when it is deployed in a first direction and releases the anchors 106 when it is moved in a second direction opposite from the first direction to retract the needle. Accordingly, the needle 100 is advanced through a subcutaneous space to the target region of the tissues. Next, the needle 100 is retracted from the suture 103 and anchors 106 for deployment.

In FIG. 9, the anchor 106 is shown in greater detail. The anchor 106 is held in position on the suture 103 by a pair of retainers 109 that may be disposed along the suture 103 on opposite sides of the anchor 106. As shown, the anchor 106 has a major portion 112 disposed coaxially with the suture 103. The anchor 106 is shown with an axial opening that receives the suture 103. A laterally extending member 115 holds the anchor 106 in position in the tissue. As shown, the laterally extending members 115 extend so that once the needle 100 is removed, the anchor members 106 grip the tissue and can be used to pull the tissue from right to left with respect to the orientation shown in FIG. 9.

The free end of the suture 103 may be anchored by tunnelizing it around the head or neck and attaching it to the contralateral suture as described above.

As an alternative, the free end of the sutures 103 may be fixed to anchoring points in the deep temporal fascia, aponeuroses, or muscles such as zygomatic, temporalis, occipitalis, frontalis, masseter, and sternocleidomastoid.

For example, an incision may be made along the temporal hairline for access to the deep temporal fascia. A dissection down to the deep temporal fascia is performed and then an elevator is used to dissect a pocket posteriorly and anteriorly past the brow in the pathway of the tunneling device. After the sutures 103 are placed into position, a French eye needle may be used to anchor the sutures to the deep temporal fascia. The sutures 103 are then tied down and the temple incision is closed.

As shown in FIG. 10, the technique may be used for the elevation and traction of facial, head and neck soft tissues, including the midface, malar tissues, eyebrows, nasolabial folds, labiomental folds, infraorbital hollows, neck soft tissues, and jowls.

As an additional alternative, the suture 103 may be provided with a coating or material, that may take the form of a fuzzy material, disposed between the anchors 106 to increase the inflammatory reaction to promote fibrosis. In the case of reabsorbable sutures, the fibrosis promoting material will hold the tissues in place after the sutures 103 have been reabsorbed.

While the invention has been described in connection with certain embodiments, it is not intended to limit the scope of the invention to the particular forms set forth, but, on the contrary, it is intended to cover such alternatives, modifications, and equivalents as may be included within the spirit and scope of the invention.

What is claimed is:

1. A percutaneous facelift device, comprising:
a needle having a lumen;
a suture disposed within the lumen, the suture having at least one anchor; and,
a plunger operatively associated with the needle such that the needle can be retracted from the suture for deployment.

2. The percutaneous facelift device of claim 1, wherein the needle has a straight tip.

3. The percutaneous facelift device of claim 1, wherein the needle has a curved tip.

4. The percutaneous facelift device of claim 1, wherein the anchor has an opening that receives the suture.

5. The percutaneous facelift device of claim 1, wherein the anchor has a major portion that is coaxial with the suture.

6. The percutaneous facelift device of claim 5, wherein the anchor has a laterally extending portion.

7. The percutaneous facelift device of claim 1, wherein the suture is reabsorbable.

8. The percutaneous facelift device of claim 1, wherein the at least one anchor is reabsorbable.

9. The percutaneous facelift device of claim 1, further comprising a material disposed between the anchors for promoting fibrosis so that the tissues are held in place after the sutures are reabsorbed.

10. A percutaneous facelift system, comprising:
a first suture having at least one anchor disposed thereon, the first suture being deployed into a target area of tissue for elevation and traction, the first suture having a first end where the anchor is disposed and having a second free end;
a second suture disposed contralateral to the first suture, the second suture having at least one anchor disposed thereon, the second suture having at least one anchor disposed thereon, the second suture being deployed into a target area of tissue for elevation and traction, the second suture having a first end where the anchor is disposed and having a second free end;
wherein the free end of the first suture is connected to the free end of the second suture.

11. The percutaneous facelift system of claim 10, wherein the free ends of the first and second suture are tunneled around the back of the head of a patient.

12. The percutaneous facelift system of claim 10, wherein the free ends of the first and second suture are tunneled around the back of the neck of a patient.

13. The percutaneous facelift system of claim 10, wherein the at least one anchor has lateral portions that extend from opposite sides of the suture.

14. The percutaneous facelift system of claim 10, wherein the at least one anchor attaches to the suture at its midpoint.

15. The percutaneous facelift system of claim 10, wherein the at least one anchor expands upon release from the needle.

16. The percutaneous facelift system of claim 10, wherein the anchor has an opening that receives the suture.

17. The percutaneous facelift system of claim 10, wherein the anchor has a major portion that is coaxial with the suture.

18. The percutaneous facelift system of claim 10, wherein the anchor has a laterally extending portion.

19. The percutaneous facelift device of claim 10, wherein the first and second suture is reabsorbable.

20. The percutaneous facelift device of claim 10, wherein the at least one anchor on the first and second suture is reabsorbable.

21. The percutaneous facelift device of claim 10, further comprising a material disposed between the anchors for promoting fibrosis so that the tissues are held in place after the sutures are reabsorbed.

22. A method of performing a percutaneous facelift, comprising:
providing a needle having a lumen, a suture disposed within the lumen, the suture having at least one anchor, and, a plunger operatively associated with the needle such that the needle can be retracted from the suture for deployment;
deploying the needle through subcutaneous space to a target area of tissue for elevation and traction;
retracting the needle while holding the suture in position with the plunger such that the suture is deployed in the target area; and, anchoring the free end of the sutures.

23. The method of claim 22, wherein the free ends of the sutures are anchored by tying a first suture to a second contralateral suture around the head of a patient.

24. The method of claim 22, wherein the free ends of the sutures are anchored by tying a first suture to a second contralateral suture around the neck of a patient.

25. The method of claim 22, wherein the free ends of the sutures are anchored to the deep temporal fascia.

26. The method of claim 22 wherein free ends of the sutures are anchored to tissue selected from the group consisting of deep temporal fascia, aponeuroses, and muscles such as zygomatic, temporalis, occipitalis, frontalis, masseter, and sternocleidomastoid.

27. The method of claim 22, wherein the suture is reabsorbable.

28. The method of claim 22, wherein the at least one anchor is reabsorbable.

29. The method of claim 22, further comprising a material disposed between the anchors for promoting fibrosis so that the tissues are held in place after the sutures are reabsorbed.
